(19) United States
(12) Patent Application Publication
(10) Pub. No.: US 2007/0060995 A1
(43) Pub. Date: Mar. 15, 2007

(54) Workflow for cardiovascular intervention

(76) Inventors: Oliver Meissner, Munchen (DE); Johannes Rieber, Munchen (DE); Thomas Redel, Poxdorf (DE)

Correspondence Address: SCHIFF HARDIN, LLP;PATENT DEPARTMENT, 6600 SEARS TOWER, CHICAGO, IL, 60606-6473, US

(21) Appl. No.: 11/208,188

(22) Filed: Aug. 19, 2005

(51) Int. Cl. A61F 2/06

(52) U.S. Cl. 623/1.11; 600/411

(57) ABSTRACT

A method is provided for a workflow for cardiovascular intervention that provides online monitoring and therapy control of the procedure. A series of steps are provided utilizing an imaging technology in combination with a therapeutic device to optimize the reliability of the treatment and minimize the negative effects on the patient. In generally, the method steps include: positioning the therapeutic device, performing the therapeutic procedure while imaging the therapeutic device, providing a low pressure inflation of the therapeutic device, imaging the results of the therapeutic procedure, and assessing the result of the therapeutic procedure. If the assessment reveals that the therapeutic procedure result is not optimal, the therapeutic procedure is performed again and the steps thereafter are performed again.

### BACKGROUND OF THE INVENTION

1. Field of the Invention

The present invention relates generally to a method for performing a cardiovascular intervention.

2. Description of the Related Art

Atherosclerotic vascular disease, the underlying pathology for ischemic heart disease, peripheral arterial occlusive disease and stroke, is the leading cause of death and disability in the modern industrialized countries. Today, after diagnosis of an occlusive disease in the coronaries/peripheral arteries, the main therapeutic approaches are PTCA/PTA (percutaneous transluminal (coronary) angioplasty) and/or stenting of the diseased vessel. Stenting may also be a future option for the treatment of so called “vulnerable” plaques.

Based on angiographic assessment, the degree of the disease, vessel dimensions and the therapeutic approach will be selected. In more complex cases intravascular ultrasound (IVUS) is used to get more detailed information from the diseased vessel segment. The next step will be the selection of the adequate therapeutic device, i.e. the material, length and diameter of the balloon and/or stent device best adapted to the vessel wall morphology and dimensions.

Normally, the inflation of the balloon is based on data given by the manufacturer of the device and the experience of the clinician performing the intervention. Today there is no monitoring of the deployment of the device with respect to the vessel wall. Moreover after dilation or stent placement the balloon catheter will be removed from the patient and a post-interventional control of the success of treatment may be performed. In case of a suboptimal result the balloon has to be introduced to the patient again to repeat the balloon expansion e.g. inside the stent with often higher pressure. This procedure is time-consuming and offers additional procedure risk and additional radiation to the patient.

Intravascular optical coherence tomography (OCT) is a new imaging modality providing histology-like cross-sectional images of coronary or peripheral arteries. The principle of OCT is analogous to B-mode ultrasound but OCT measures the back-reflection of near coherent infrared light instead of acoustical waves. With the high energy of light, OCT is able to achieve a spatial resolution of 10-20 μm, which is about 10 times higher than that of any other clinically available diagnostic imaging modality. Similar to IVUS, the known standard of reference for in vivo diagnostics, OCT allows for real-time imaging of vessel dimensions and vessel wall morphology, but with its much higher resolution, OCT is able to discriminate important structures like the fibrous cap or the lipid core of an atherosclerotic plaque. This additional information might have an important impact on planning and performing interventional procedures.

Furthermore, the OCT imaging device is very small in diameter (about 0.014 inch as compared to the IVUS probe with size of 2.7 French in diameter), making it possible to advance into the lumen of a diagnostic or interventional catheter and even pass through high-grade stenosis or small side branches.

An important advantage of OCT is the much lesser susceptibility to artifacts caused by metallic material like stents or by calcified plaques. Stent apposition to the vessel wall as well as stent symmetry and unfolding can be better delineated with OCT than with IVUS.

Even more, most balloon catheters used for PTCA/PTA or stent expansion are made out of materials which are translucent in the wavelength range used by OCT (typically 1300 nm).

The above mentioned allows for an online monitoring of interventional procedures like balloon dilation or stent deployment with OCT. This advanced use of OCT leads to an improved workflow of interventional procedures with possibly better long-term results regarding re-stenosis or occlusion of the affected vessel.

New methods which will provide similar information like IVUS are the “Optical Coherence Tomography” (OCT), “Optical Frequency Domain Tomography” (OFDI) and “Spectral Domain OCT” (SD-OCT).

Until now, interventional cardiologists and radiologists primarily rely on pre- and post-interventional information acquired with angiography or IVUS.

Digital subtraction angiography (DSA) as a two dimensional luminogram of the vessel is not able to discriminate different plaque types. This also holds for three dimensional reconstructions.

Angiography and sophisticated analytical methods (QCA, IC3D) are used for determination of length and diameter of the vessel segment being treated and selection of the treatment device.

Using contrast agents for inflation of the balloon allows for a two dimensional projective assessment of the success during treatment. This again is limited by the two dimensional nature of this view and by the fact that the vessel wall can't be seen by this way.

After intervention an assessment of the success is done by visualization of the improved flow through the dilated or stented artery. In case of a stent placement, an important fact is the complete apposition of the stent struts to the vessel wall to avoid subacute stent thrombosis. Stent apposition to the vessel wall can not exactly be estimated with angiography.

Using IVUS allows for a detailed assessment of the vessel segment as well as of the composition of the vessel wall and is used for a more detailed planning and for post-interventional control of the therapeutic approach.

Today the IVUS catheter is very bulky and efforts to directly guide the dilation process with the help of a combined IVUS/stent device were not promising.

In case of post-interventional assessment of the apposition of the stent struts the accuracy of this method is very limited due to the artifacts induced by the metallic struts and the limited spatial resolution of IVUS (100 μm as compared to OCT with 10-20 μm).

For post-interventional assessment, the therapeutic delivery catheter has to be removed outside the treated lesion. In case of a suboptimal result, the balloon has to be introduced into the lesion again to repeat the balloon expansion e.g. inside the stent with somewhat higher pressure. This means an additional risk to the patient and is much more time-consuming.

An online guiding and monitoring of interventional procedures is not known due to the above mentioned limitations of IVUS or angiography. This is also true for assessment of the success of a therapeutic procedure without removing the delivery catheter. This invention will provide a feasible approach and workflow to overcome these limitations.

### SUMMARY OF THE INVENTION

The present invention provides a workflow for cardiovascular intervention that provides online monitoring and therapy control of the procedure. A series of steps are provided utilizing an imaging technology in combination with a therapeutic device to optimize the reliability of the treatment and minimize the negative effects on the patient. In generally, the method steps include: positioning the therapeutic device, performing the therapeutic procedure while imaging the therapeutic device, providing a low pressure inflation of the therapeutic device, imaging the results of the therapeutic procedure, and assessing the result of the therapeutic procedure. If the assessment reveals that the therapeutic procedure result is not optimal, the therapeutic procedure is performed again and the steps set forth above are performed again.

Thus, the therapeutic procedure is monitored as to its result without requiring removal of the dilation balloon of the therapeutic device.

### BRIEF DESCRIPTION OF THE DRAWINGS

FIG. 1 is a flow chart showing the steps in an embodiment of the present method.

### DETAILED DESCRIPTION OF THE PREFERRED EMBODIMENTS

The present method provides a preferred workflow for online monitoring the therapy control. In a preferred embodiment, the therapeutic procedure for interventional cardiology includes the following consecutive steps.

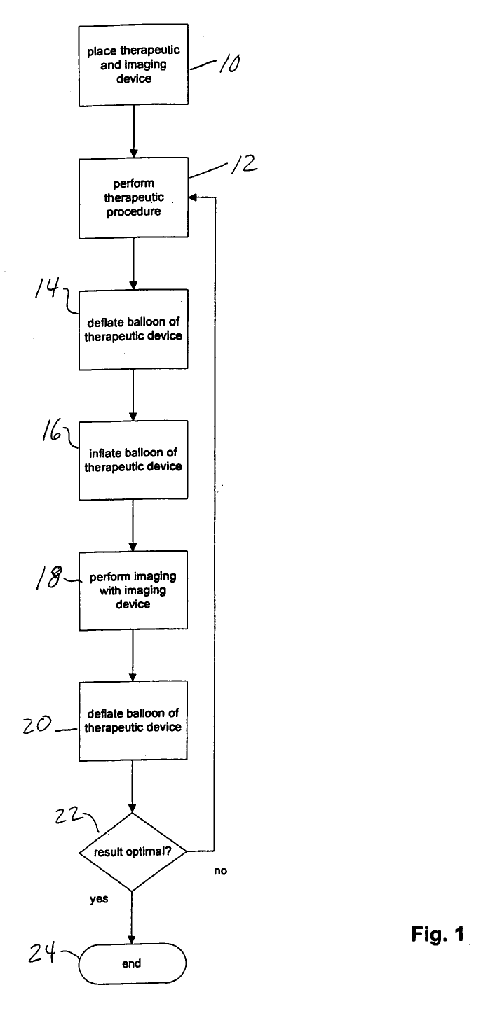

1. Placement, or positioning, of a combined therapeutic device which offers the possibility to monitor the therapeutic procedure under angiographic view. In FIG. 1, this is shown as step 10. The therapeutic device is generally positioned in the afflicted artery. The device of one embodiment is a PTCA (percutaneous transluminal coronary angioplasty) balloon catheter or a stent delivery catheter in combination with an OCT (optical coherence tomography) catheter. Other embodiments of the invention include, but are not limited to, the therapeutic device being one of the devices on the following list: PTA balloon catheter, a bare metal stent, a drug eluting stent, a bioresorbable stent, or a drug eluting bioresorbable stent. Other devices are possible in the present method.

2. Proceeding with the therapeutic procedure and simultaneously imaging the therapeutic device with respect to the vessel wall using an invasive imaging technique like OCT, as shown at step 12. During this procedure, the dilation balloon or another low pressure balloon attached to the device occludes the vessel and removes the blood from the field of view in order to make OCT imaging possible.

3. As an optional step 14, deflating the balloon after the therapeutic procedure to allow blood flow to the heart muscle.

4. Providing a low-pressure inflation 16 of the balloon with a pressure sufficient to achieve a bloodless situation in the dilated/stent area. This enables the OCT device to control the interventional procedure.

5. Starting a pullback with the noninvasive imaging device (e.g. the OCT) in the dilated area/stented area to image the area, as indicated at step 18, and assess the result of the therapeutic interventional procedure, e.g. to control stent unfolding or apposition to the vessel wall.

6. As an optional step 20, deflating the balloon after the assessment procedure to allow blood flow to the heart muscle.

7. Determining if the result is optimal or less than optimal at step 22. If an optimal result is obtained, ending the procedure at 24. In case of a suboptimal result, return to step 12 and inflating the balloon again with a pressure sufficient for therapeutic compliance. The steps following step 12 are again performed, the optional steps 14 and 20 again being optional.

This allows for an online monitoring and a control of the procedure without removing the dilatation/delivery balloon.

Of course this specific workflow described above is only a part of an overall workflow of an interventional procedure like balloon dilation or stent placement in a coronary artery.

The present method offers also a detailed workflow for online guiding, monitoring and control. In detail, the single steps of the workflow include:

1. A localization of the lesion to treat (the target lesion) is performed, either with angiography, digital subtraction angiography (DSA), magnetic resonance angiography (MRA) or computer tomography angiography (CTA).

2. A characterization of the target lesion (plaque composition, quantification of lumen and vessel wall dimensions) by a invasive cross sectional imaging technique like, OFDI, OCT, IVUS is carried out.

3. An optional determination is made of the plaque burden, degree of stenosis with the help of a dedicated software tool based on the data received under the characterization step 2.

4. Choosing the optimal device (balloon size, stent device, and the like) on the basis of the data received under steps 2 and 3.

5. Performing offline planning of the interventional procedure based on the data received under steps 2, 3 and 4.

6. Advancing the device into the target lesion under angiographic guidance.

7. Providing an online intervention monitoring and control as described in the workflow steps described above and shown in FIG. 1.

8. Performing the optional steps of proceeding on additional lesions by reentering the workflow at step 2.

9. Generating final documentation including data and images received under steps 2, 3, 4 and 7.

This workflow for the monitoring and controlling of the therapeutic procedure shows two main advantages:

The therapeutic procedure can optimally be adapted to the individual target lesion by adapting the pressure for the balloon inflation to the morphologic and geometricry reaction of the vessel wall. Therefore, as minimal damage to the vessel wall as necessary can be achieved with a maximum of therapeutic success. The procedure described in the workflow might lead to better longterm results due to optimized intervention.

In case of a suboptimal therapeutic result, an immediate control without removing the therapeutic catheter reduces the risk and radiation to the patient and shortens the whole procedure time.

A dedicated workflow may limit errors adherent to the complex application flow of an interventional procedure.

Although other modifications and changes may be suggested by those skilled in the art, it is the intention of the inventors to embody within the patent warranted hereon all changes and modifications as reasonably and properly come within the scope of their contribution to the art.

We claim:

1. A method for performing a cardiovascular intervention, comprising the steps of:
positioning a combination therapeutic device and imaging device in a cardiac artery;
performing a cardiovascular therapeutic procedure at a therapy site including inflating a balloon of the therapeutic device;
performing a low-pressure inflation of the balloon;
imaging the therapy site with the imaging device;
determining whether a result of said cardiovascular therapeutic procedure meets an acceptability criteria;
performing said cardiovascular therapeutic procedure again if said determining step determines that a result is below an acceptability criteria.

2. A method as claimed in claim 1, further comprising the step of:
deflating the balloon of the therapeutic device after said step of performing the cardiovascular therapeutic procedure.

3. A method as claimed in claim 1, further comprising the step of:
deflating the balloon of the therapeutic device after said step of determining whether the result meet the acceptability criteria.

4. A method as claimed in claim 1, wherein said imaging device is an optical coherence tomography device.

5. A method as claimed in claim 1, wherein said therapeutic device is a stent delivery catheter.

6. A method as claimed in claim 5, wherein said step of determining the result monitors stent unfolding.

7. A method as claimed in claim 1, wherein said therapeutic device is a balloon catheter.

8. A method as claimed in claim 7, wherein said balloon catheter is a percutaneous transluminal coronary angioplasty catheter.

9. A method as claimed in claim 7, wherein said step of determining the result monitors a position of a vessel wall.

10. A method as claimed in claim 1, wherein said step of performing the low pressure inflation of the balloon inflates the balloon sufficiently to achieve a bloodless state in the artery.
